(19) United States
(12) Patent Application Publication
(10) Pub. No.: US 2019/0320268 A1
(43) Pub. Date: Oct. 17, 2019

(54) SYSTEMS, DEVICES AND METHODS FOR EXECUTING A DIGITAL AUDIOGRAM

(71) Applicant: Listening Applications LTD, Jerusalem (IL)

(72) Inventors: Yoav Blau, Tel Aviv (IL); Tomer Shor, Jerusalem (IL); Yonatan Roth, Jerusalem (IL); Sabrina Levi, Jerusalem (IL)

(21) Appl. No.: 16/381,466

(22) Filed: Apr. 11, 2019

Related U.S. Application Data
(60) Provisional application No. 62/655,845, filed on Apr. 11, 2018.

(51) Int. Cl. H04R 25/00; G10K 11/175; A61B 5/12; A61B 5/00

(52) U.S. Cl. CPC H04R 25/453; G10K 11/175; A61B 5/123; A61B 5/6898; H04R 2460/01; A61B 5/743; A61B 5/0002; A61B 5/746; G10K 2210/1081; A61B 5/7405

(57) ABSTRACT

A system is disclosed for providing optimized audio output. The system comprises a device, such as a mobile phone connectable to one or more earphones. The device may comprise a computing platform having a processor; an audio unit configured to generate one or more output signals of arbitrary amplitude to the one or more earphones at least one microphone configured to record the power level of the outputs signals and calculate a proportionality constant for each frequency of the output signals; and wherein the processor is further configured to: analyze the proportionality constant for each frequency of one or more feedback signals from the one or more earphones to yield calibration data; adjust the amplitude or frequency based at least on the calibration data to calibrate the device; generate one or more audiograms resulted by conducting a hearing test using the calibrated device; and adjust the device power level according to the received one or more audiograms.

### CROSS-REFERENCE

The present application claims the benefit of U.S. Provisional Application Ser. No. 62/655,845 filed on 11 Apr. 2018, entitled “SYSTEM AND METHOD FOR REMOTE AUDIOLOGY TESTING”, which is incorporated herein by reference in its entirety.

### FIELD OF THE INVENTION

The present invention relates in general to methods, systems and devices useful in Audiology, and specifically relates to audiometric testing and usages thereof.

### BACKGROUND OF THE INVENTION

Hearing tests are generally conducted by hearing institutes or laboratories, and are performed using specialized medical devices such as audiometers, special headphones and sealed rooms. The most common type of audiometer generates pure tones, with varying amplitudes as chosen by a human operator, typically a hearing specialist, and delivered to the subject's ears through the headphones. During testing, the subject indicates that a tone was heard by pressing a feedback button or by a visual signal to the operator. The audiometer enables the operator to produce an audiogram, describing the subject's hearing acuity. The prior methods and systems for performing the hearing tests can be less than ideal in at least some respects. Presently, the art requires expensive equipment and an expert checker which must use the specialized medical devices, for example as part of the hearing test to get accurate results. This requires substantial expense and time, thereby preventing many end users from receiving adequate hearing testing, or hearing surveillance, which could help to identify and prevent hearing deterioration. A practical method for tracking such deterioration was suggested by the Occupational Health Service for the Northern Ireland Civil Service (MacLurg et al., 2004). The analysis they provide may serve as guidelines to produce alerts or advice regarding the state of a user's hearing. However, this requires that the user undergoes hearing tests periodically.

It would be highly advantageous to have a system or method that could enable conducting of remote hearing tests using non-specialist equipment, with high accuracy, for example at home or out of a laboratory, thereby avoiding the inconvenience and/or the expense of going to doctors and/or clinics and using standard devices such as a personal computer (PC), tablet, smartphones, and earphones, for convenient, accessible, user-friendly, efficient and economical operation.

### SUMMARY OF THE INVENTION

There are provided, in accordance with embodiments, an improved audiology testing systems, devices and methods which may be performed using standard devices such as a laptop, tablet computer, media console, personal digital assistants or smart phone, or any other sort of device having a processor and audio units for example a user's mobile phone for performing a clinical hearing test (e.g. remote audiology testing).

In some embodiments, the clinical hearing test may be performed without expert intervention and without a medical device.

In some embodiments, the audiology testing system may include components for facilitating remote audiology for example, a mobile device, headphones; and a microphone such as a sound level meter (SLM).

In some embodiments, a system is provided for providing optimized audio output, the system comprising: a device connectable to an earphone, the device comprising a computing platform having a processor; an audio unit configured to generate one or more output signals of arbitrary amplitude to the earphone; a microphone configured to record the power level of said outputs signals and calculate a proportionality constant for each frequency of said output signals; and wherein the processor is further configured to: analyze the proportionality constant for each frequency of one or more feedback signals from said earphone to yield calibration data; adjust the amplitude or frequency based at least on the calibration data to calibrate the device; generate one or more audiograms resulted by conducting a hearing test using the calibrated device; adjust said device power level according to said received one or more audiograms.

In some embodiments the processer is configured to calculate a fractional amplitude coefficient for each of said feedback signals for providing said calibration data.

In some embodiments the hearing test is executed by said processor according to a state deterministic automaton.

In some embodiments the hearing test is based on the Hughson-Westlake technique.

In some embodiments the audiograms are applied to one or more selected remote devices having a processor, said processor is configured to adjust the selected device audio power based on the audiograms.

In some embodiments the device is a mobile communication device comprising wireless communication circuitry to communicate with a remote server, and wherein the processor comprising instructions to transmit the audiogram to the remote server.

In some embodiments, in response to the instructions the audiograms are further integrated in the remote server database for adjusting an audio output control of one or more contents or application in the remote server database in accordance with the integrated audiogram.

In some embodiments the audiograms are applied to a communication layer of communications provider of the remote server for adjusting an audio output control of the communication layer of communications provider in the remote server database in accordance with the integrated audiogram.

In some embodiments the content or application is selected from the group consisting of: YouTube, iTunes, Netflix, audiobooks, radio stations, conferencing software.

In some embodiments the earphone is selected from a group consisting of: noise cancellation earphones, wireless earphones, wired earphones.

In some embodiments the microphone is a sound level meter (SLM).

In some embodiments the audiograms are generated in a digital format.

In some embodiments the audiograms comprise personal preferences of said user.

In some embodiments the audiograms are shared with other devices or applications.

In some embodiments the other devices or applications are selected from the group consisting of: computers, PCs, mobile devices, televisions YouTube, Netflix, cable TV, Spotify, Apple music, online radio stations, games.

In some embodiments the audiograms are shared with other devices via a network server.

In some embodiments the audiograms are applied to a cloud-based Conference Call program, to optimize output to conference call users.

In some embodiments the audiograms are applied to digital assistant devices.

In some embodiments the digital assistant devices are one or more of Alexa, Seri, personal robots, guides, assistants.

In some embodiments the audiograms are applied to broadcast radio.

In some embodiments the audiograms are applied an audio output channel to provide audio output adapted to a user's accent or dialect.

In some embodiments the audiograms are integrated into a processor of noise cancelation earphones for converting said noise cancelation earphones to audio enhancing devices or hearing aid devices.

In some embodiments the conversion comprises filtering audio signals received at the noise cancelation earphones and amplifying audio signals yield a personalized audio output by the noise cancelation earphones.

According to some embodiments, a method is provided for providing optimized audio output using a device comprising an audio unit and a processor, and wherein the device is further connectable to an earphone, the method comprising: performing a hearing test to a user using the device, the hearing test comprising generating signals at selected frequencies and hearing levels and recording the user feedback; generating a digital audiogram profile based on the hearing test; transmitting said digital audiogram to the device or to a remote server; and converting the digital audiogram to an audio filter; adjusting the device audio output to yield an optimized audio output.

In some embodiments, the method includes converting the digital audiogram to an audio filter comprises shaping input signal so as to be amplified by a magnitude equivalent to the audiogram's gain level for each tested frequency, or some other frequency response derived from said digital audiogram, for example 50% the gain levels.

In some embodiments, the method includes adjusting the device audio output is configured by applying the audio filter, or other filters which may be beneficial for the user, such as noise reduction, or band-pass filtering.

According to some embodiments, a server-based audiogram analysis engine system is provided, the system comprising: a remote server, the remote server is in communication with a database and a remote processor; a plurality of remote devices connectable respectively to a plurality of earphones, wherein each of the plurality of remote devices comprises: a computing platform having a processor; an audio unit configured to generate respectively one or more output signals of arbitrary amplitude to said plurality of headphones; wireless communication circuitry to communicate with the remote server; and wherein each of the remote devices is configured to: perform a hearing test to one or more users, the hearing test comprises: generating one or more output signals of arbitrary amplitude to yield an audiogram profile respectively for each of the users; transmit the audiogram profile to said remote server, wherein the remote server comprises instructions to: analyze the audiogram profiles; convert the audiogram profiles to yield a personalized audio filter for each of a plurality of remote devices.

In some embodiments the analysis comprises generating one or more alerts.

According to some embodiments, a noise-cancelling headphone is provided that is connectable to a portable computing platform having a processor, the noise-cancelling headphone comprising: an electro-acoustic transducer converting ambient noise into a noise signal; a cancel signal generator generating and outputting a cancel signal to eliminate the noise from the noise signal; and a speaker unit outputting an audio signal and the cancel signal, wherein the processor is configured to: receive one or more audiograms; convert the audiograms as a function of the frequency in which a hearing gain level was established for the user; filter the cancel signal based on said function to transform the noise-cancelling headphone to a hearing aid.

In some embodiments the audiogram is a digital audiogram.

In some embodiments the digital audiogram is obtained by performing a hearing test using the above described system.

A machine-readable non-transitory medium is herein provided, encoded with executable instructions for transforming a noise-cancelling headphone to a hearing aid, the instructions comprising code for: converting one or more audiograms of a user of the noise-cancelling headphone as a function of the frequency in which a hearing gain level was established for the user; and filtering a cancel signal based on the function to transform said noise-cancelling headphone to a hearing aid.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic system diagram depicting an audiology system, in accordance with embodiments;

FIG. 2 is a flowchart of a method for performing an audiometric test, in accordance with embodiments;

FIG. 3 is a flow diagram of a testing protocol for a given tone, according to the prior art;

FIG. 4 is a hearing test designed to be conducted by a state machine, without human judgment being used, by a deterministic automaton, according to the prior art;

FIG. 5 is a graphic illustration of an audiogram method, where the audiogram is the frequency response of a human ear, in accordance with embodiments;

FIG. 6 is an illustration of the Audiogram Aggregation model based on the assumption that the user has one true hearing level per frequency, or audiogram A(f), obstructed by noise factors, in accordance with embodiments;

FIG. 7 is a schematic system diagram depicting a plurality of applications based on a digital audiogram connected to the cloud, in accordance with embodiments;

FIG. 8 is a flow diagram for execution of a digital audiogram in an audio playback system, in accordance with some embodiments;

FIGS. 9A-9C are a series of work flow diagrams showing examples of optimizing audio output with noise cancellation headphones, according to the principles of the present invention; and

FIG. 10 shows a computer system suitable for incorporation with the methods and apparatus in accordance with some embodiments.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

There is provided, in accordance with an embodiment, an apparatus, system, and method configured and operable to perform audiologist functionalities on a computing device such as remote computing device or mobile device, including for example smartphones, laptops or notepads, standard headphones, and the like.

Non-limiting embodiments of the present invention include systems, devices methods and/or means for facilitating clinical audiometric testing at home or out of a laboratory, thereby avoiding the inconvenience and/or the expense of going to doctors and/or clinics to achieve the same. The increased accessibility will increase the number of people being tested and remove barriers for many with intermediate hearing loss. In this way, many people who are in need of being tested already have the sufficient hardware to perform the hearing test on their person. Furthermore, there are provided embodiments to facilitate recurring periodic tests, thereby enabling testees to track their hearing and detect deterioration in their hearing abilities as soon as possible.

The term “gain level” as used herein and through the specification and claims should be understood to encompass dB HL (as defined for example by ANSI (1996)) and “gain jumps” in dB (decibels).

The term “headphones” or “earphones” as used herein and through the specification and claims should be understood to encompass earphones such as a sound receiver which may be placed in or over the ear or held over the ear by a band or headset.

The term a “Sound Level Meter” (SLM) as used herein and through the specification and claims should be understood to encompass an acoustic measurement unit such as a hand-held device including a microphone. For a given configuration, of hardware and software (e.g., system volume setting, digital signal amplitude etc.), SLM may set the reference power level in standard SPL units, which is Sound Pressure Level. Specifically, the SLM may be a microphone configured to handle a broad spectrum well and may be calibrated to assign different weights to each frequency band. In some cases, the audio it records is accumulated periodically to produce one number, the sound level.

In accordance with embodiments, the SLM may be used to calibrate a remote testing system (e.g. the user's mobile device such as the user's smartphone and headphones), therefore providing a virtual hearing aid. For example, in some cases, the SLM may be pressed against the user's headphones and record the power omitted when a tone is played by the user's device (e.g. smart phone) in each frequency at some amplitude level. This way, for each frequency, the proportionality constant is measured and determined. In some cases, following calibration, a testing setup produces tones of definite gain level, in HL (hearing level) units, enabling to conduct a precise audio test.

According to some embodiments, a remote audiology testing method and system is provided comprising calibrating a user's system including a mobile device such as a mobile phone connectable to one or more earphones and using the calibrated system for performing a remote hearing test.

In some cases, the mobile device is connectable to at least a portable computing platform having a processor, an audio unit and a microphone.

In some cases, the microphone may be an SLM configured to calibrate the user's system (e.g. mobile device).

In some cases, an SLM is not required, rather the approximate frequency response of the user's hardware may be used, based on using calibration transfer.

In some embodiments, a method for executing a “calibration transfer” may include calibrating (e.g. the proportionality constant, or gain, per frequency) for a new type of device, subtracting (e.g. in logarithmic scale) the frequency response of one device, and adding (e.g. in logarithmic scale) the frequency response of another device, such that the new device can be calibrated, without ever having tested or used for such a test, wherein the new device calibration is hereinafter referred to as a “calibration transfer”, and wherein, if all devices of the same type are similar, then the calibrating for one such device provides a calibration for all similar devices.

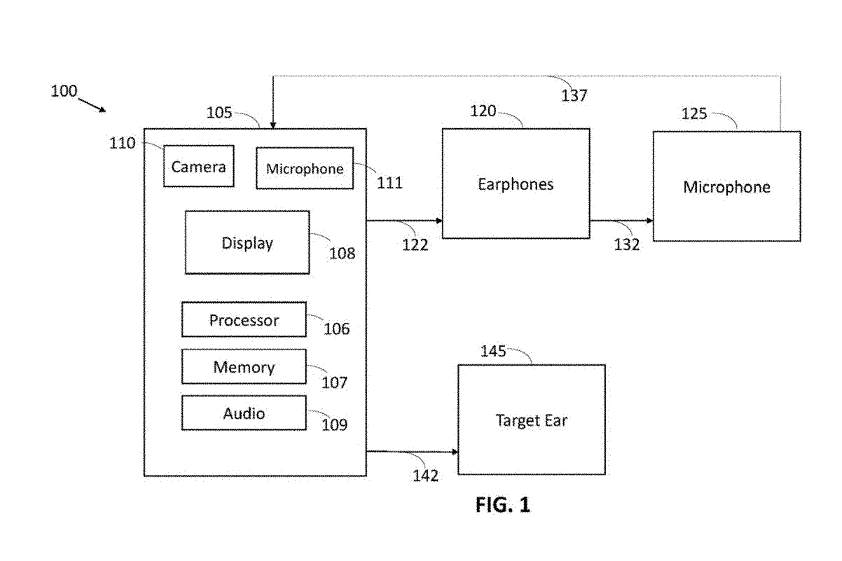

Reference is now made to FIG. 1, which is a schematic system diagram depicting components in an audiology system 100, in accordance with embodiments. The audiology system 100 is configured to facilitate remote audiology calibration using standard typical computer devices for example a portable device (e.g. mobile phone), headphone and microphone and may be further used or configured as a virtual hearing aid for conducting a precise audiometric test. A virtual hearing aid may be defined, in accordance with embodiments, as usage of non-specialized devices and systems such as standard hearing hardware and/or software to compensate for hearing loss or complement hearing difficulties, for example, changing audio output in accordance with a listener's hearing profile.

In some cases, the remote audiology system 100 may include a computerized device 105 such as a portable computing platform, headphones or earphones 120 and a microphone 125.

The device 105 may comprise, for example, desktop, laptop, or tablet computer, media console, personal digital assistants or smart phone, or any other sort of device having a processor and audio units. In some cases, the device 105 is configured to be in communication with a network, video and audio interfaces and computing capabilities needed to interact with server. By way of example, device 105 may be a mobile device having a processor 106, memory 107, video display 108 and audio unit 109 including an audio input and output configured to generate and receive one or more audio signals, along with a video camera 110 and microphone 111 for recording.

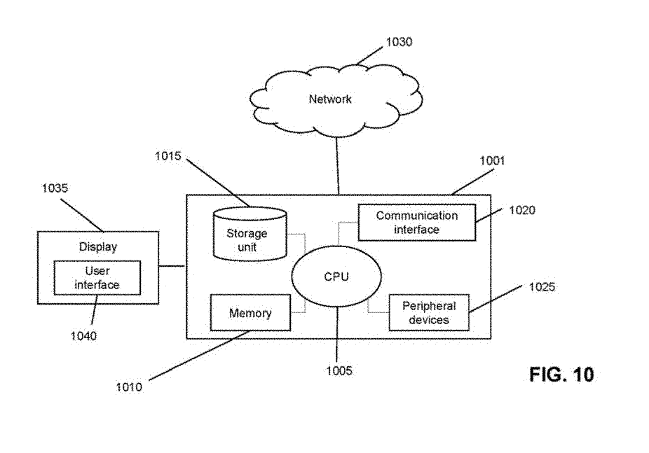

In some cases, the device 105 may be or may include units of system 1001 illustrated in FIG. 10.

According to some embodiments, the headphones 120 may be wired headphones or wireless headphones such as a Bluetooth™ headphones, for receiving audio input 132 from device 105.

According to some embodiments, the microphone 125 may be or may include a sound level meter (SLM).

In some cases the microphone 125 may include a measuring module for example in the form of software or firmware component configured to use an audio recorded by the microphone 125 to measure sound pressure level (SPL).

In operation, for a given configuration, of hardware and software (e.g., system volume setting, fractional tone amplitude etc.), the microphone 125 (e.g. SLM) is configured to receive one or more tones 132 used to calibrate the audiology system 100 by setting a reference power level (for example in standard SPL units), the reference power level may be for example P0=20 μPa=0 dB SPL, as defined for example by IEC 60027-3:2002, and providing a coefficient magnitude 137 which is needed to amplify in each frequency to get the standardized hearing levels. Following the calibration process, the device 105 produces one or more tones 142 of definite gain level, for example in HL (hearing level) units towards a target ear 145, for conducting a precise audiometric test. The right-pointing arrows 122, 132 indicate the signal propagation, and the left-pointing arrow 137 going back from the microphone 125 to the device 105 provides the measured values (feedback).

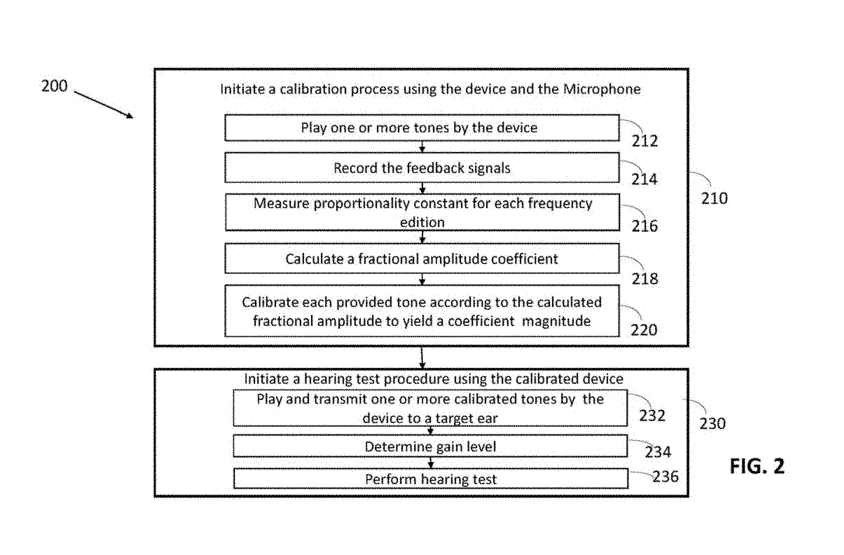

FIG. 2 is a flowchart 200 of a method for performing an audiometric test, such as an audiometric self-test using a standard computerized system such as system 100 including a mobile device (e.g. mobile phone) connectable to a headphone and a microphone, in accordance with embodiments. The standard computerized system is configured as a virtual hearing aid which may provide the mobile device user an optimal hearing experience anywhere he goes. At step 210 a calibration process is initiated, for example by the device 105 processor 106 to set the reference sound power level (e.g. in standard SPL units). The calibration process may be operated for example using the microphone 125, and/or an SLM. In some cases, the calibration includes at step 212 playing one or more tones by the device audio unit 109, for example near the headphones 120. Specifically, the calibration may include transmitting from the device 105 to the headphones 120 one or more output signals such as pure tone signals, or signals including tones slightly modulated around a carrier frequency, or modulated by an envelope signal, of arbitrary amplitude (e.g. signals 122 of FIG. 1). In some cases, the output signals may be “warbled” tones, which can take different shapes and may not be strictly speaking “pure” tones. In some cases, the tone may be provided in each frequency at some amplitude level, for measuring for each frequency at some amplitude level. At step 214 the power omitted (e.g. feedback signals) by playing the tone in each frequency is recorded, for example at the device 105, and at step 216 the proportionality constant for each frequency is measured. In some cases, the measuring step is performed by recording feedback signals from the headphones membrane, treating it as a microphone or by the SLM. For example, this may be done by creating an acoustic interface between the microphone 125, or SLM, and the headphones 120. Optionally, the signal is weighted properly (e.g. by A-weighting, as defined for example by international standard IEC 61672:2003) and then its power is evaluated, which is linearly proportional to the signal's sound pressure level. At step 218 a fractional amplitude coefficient is calculated, for example by the device processor 106, and at step 220 each provided tone is calibrated according to the calculated fractional amplitude to yield the coefficient magnitude which is needed to amplify in each frequency to get the standardized hearing levels in dB HL, as defined for example by ANSI (1996).

Following the system's calibration procedure, the hearing testing procedure is initiated at step 230. Generally, the testing process includes producing tones of definite gain level, in HL (hearing level) units, enabling to conduct a precise audiometric test. Specifically, the hearing test includes at step 232 playing one or more calibrated tones transmitted from the device 105 to a target ear 145, for example via the headphone 120. At step 234 a gain level which is required for providing an output tone which includes the required power in hearing level (HL) standard scale is determined to enable conducting a professional level hearing test. At step 236 a hearing test is performed.

Advantageously the system 100 and method 200, may be configured as a virtual hearing aid which provides to the user an optimal hearing experience in multiple locations or environments where the user may be located, for example the user may conduct a test in his home or office, provided that the surrounding noise is lower than the user's threshold hearing, i.e. a quiet environment, as perceived by the user.

In some cases, the Virtual Hearing Aid (VHA), and the SLM, may be attached, by an acoustic interface, in a manner that persistently delivers partial or full power emitted from the VHA via the headphones to the SLM.

For example, system 100 may produce a specific gain Gₒᵤₜ^{dBHL} (in dBHL units) to be delivered to subject ear, and the fractional amplitude of the produced sine wave would be:

Aₒᵤₜ^{dB}=Gₒᵤₜ^{dBHL}+T_{HL}(f)−P_{SLM}^{dBSPL}(f)+G_{c}

Where: f is the tone frequency, P_{SLM}^{dBSPL} is the SLM power reading for a tone produced by the system 100 audio unit with fractional amplitude G_{c}, and T_{HL} is a standard table converting dBSPL to dBHL units.

In some cases, an arbitrary global gain level G_{c} is chosen, which permits recording emitted power by the microphone (e.g. SLM) for all or almost all target frequencies. With this gain level as fractional amplitude coefficient, the power is then recorded for tones in all target frequencies (for example 125, 250, 500, 750, 1000, 1500, 2000, 3000, 4000, 6000, 8000 Hz), for each ear (left, right). According to some embodiments, the tones may be audio sample arrays of the shape G_{c} sin(2πft).

In some cases, for a given frequency f a pure tone sine function with amplitude 1 is generated sin(2πft). This tone may be amplified by a constant gain factor Gₜₑₛₜ. The signal is then multiplied by the test gain Hₜₑₛₜ that achieves minimum audibility. The signal is multiplied by the system's volume factor V (matching the current state of the device 105) and is then produced by the soundcard 109 H_{sc} and transmitted to the headphones 120 Hₕₚ.
In some cases, a subject threshold power (hearing level) in sound pressure level (SPL) Pₜₕ^{SPL} can be expressed by:

Pₜₕ^{SPL}(ω)=GₜₑₛₜH_{test-LR}(ω)H_{sc}(ω)H_{hp-LR}(ω)L_{ear-LR}(ω) (1)
where L_{ear-LR} represents the subject's hearing loss as a function of excitation frequency.
Hearing impairment can be defined in this framework as having threshold hearing higher than normal threshold hearing for some frequency. It is therefore assumed that the same values of Pₜₕ are desired for all people, and hearing aids aim to set

Pₜ_{ₕ}(ω)=Pₜ_{ₕ}(ω)L_{ear-LR}(ω)H_{aid-LR}(ω), (2)
where the left-hand part of eq. (2) represents the threshold hearing of an ideally hearing human ear, and the right-hand part represents the threshold hearing of a person whose hearing is impaired by the loss function L_{ear-LR} and corrected by a hearing aid supplying the gain function H_{aid-LR}(ω). One aims to recover the latter and name this function the audiogram.
Using the calibration setup, one measures the output power from the sound level meter P_{SLM}^{SP}(ω) as a function of the source signal, amplified by G_{calib} with test system volume set to V.

P_{SLM-LR}^{SPL}(ω)=G_{calib}VH_{sc}(ω)_{Hhp-LR}(ω) (3)
Putting (3) into (1) one gets

Pₜ_{ₕ}^{SPL}(ω)=GₜₑₛₜH_{test-LR}(ω)G⁻¹_{calib}P_{SLM-LR}^{SPL}(ω)L_{ear-LR}(ω). (4)
One uses a conversion table T_{HL}(ω) to translate power in SPL to hearing level HL, such that

P^{HL}(ω)T_{HL}(ω)=P^{SPL}(ω) (5)
This table has positive gain values, e.g. T_{HL} (1 KHz)=7.5 dB.
One can now rewrite eq. (4) in HL and note that P^{HL}ₜₕ is, by definition, unity.

P^{HL}ₜₕ=GₜₑₛₜH_{test-LR}(ω)G⁻¹_{calib}T⁻¹_{HL}(ω)P^{SPL}_{SLM-LR}(ω)L_{ear-LR}(ω)≡1 (6)
Using eq. (2) one identifies the audiogram to be

H^{HL}_{aid-LR}(ω)=T⁻¹_{HL}(ω)P^{SPL}_{SLM-LR}(ω)G⁻¹_{calib}GₜₑₛₜH_{test-LR}(ω) (7)
Expressed in decibels, the audiogram A_{LR} takes the form:

A_{LR}=−T_{HL}(ω)+P^{SPL}_{SLM-LR}(ω)−G_{calib}+Gₜₑₛₜ+H_{test-LR}(ω) (8)
To perform calibrated audio tests, and refrain from producing tones below 0 dBHL and above some maximum (for subject safety, and to keep within hardware dynamic range), it is required that the system produces tones of calibrated power. To produce a tone of specific gain (in dBHL) K_{HL}, the corresponding amplitude K_{system} would be:

K_{system}=K_{HL-LR}(ω)+G_{calib}−P^{SPL}_{SLM-LR}(ω)+T_{HL}(ω) (9)
Where one used eq. (8), replacing the audiogram value A_{LR} with the chosen tone level K_{HL} and the corresponding test gain and gain factor Hₜₑₛₜ+Gₜₑₛₜ with the output gain K_{system}. The calculated amplitude is in dB units. Its reference level depends on the hardware and volume settings. It is inconsequential as long as the setup remains unchanged.

It may be noted that, in accordance with some embodiments:
1. The system, such as system 100 (software, OS, soundcard, headphones) should remain the same throughout testing and calibration process. All volume controls are set to 50% for uniformity and a wide dynamic range;
2. It is assumed that the hearing test was conducted in a silent ambience and that calibration was done far above ambient noise, thus all noise factors are neglected; and
3. It is assumed that the (for example all) transmission functions are linear in power and do not change their behavior with respect to frequency. This is known to be approximately true for human hearing, and remains true for the system within its dynamic range. Otherwise, functions of frequency need to be replaced with functions of frequency and input power, and the calibration process becomes more involved.

In further embodiments, the system and method may include calibrating a system including for example standard devices, for example a virtual hearing aid based upon specialist usage of devices and systems such as device 105. The calibration is needed for controlling and/or adjusting the device's output signal power and configure the device to perform clinical grade hearing tests.

In accordance with some embodiments, the calibration process may include: playing a digital audio signal (e.g. binary signal) including a list of integer amplitude values; a 16 bit pcm (or raw file), for example, has integer values from −2¹⁵ to 2¹⁵; the soundcard converts this binary signal to an analog electric signal proportional to the amplitude values; the proportionality constant is different between different systems; the volume controls this constant.

Further, in some cases for calibration system or method, the microphone can be configured to perform the function of a Sound Level Meter (SLM), if it features a wideband frequency response. This is done by filtering the signal to assign different weights to different frequency bands (e.g. by A/B/C/D/Z-weighting, as defined by international standard IEC 61672:2003). The filtered audio is accumulated continuously or periodically, for example every second, to produce a measurement of the total received signal in units of SPL. The SLM may be used to calibrate the system, by pressing it against the headphones and recording the power omitted when a tone is played in each frequency at some amplitude level. This way, for each frequency, the proportionality constant is known.

In this way, the SLM can be used to get the proportionality constant per frequency; for calculating how much to amplify in each frequency to get the standardized hearing levels; and at this stage the calibrated tones can be played.

According to some embodiments, if all devices of the same type are similar, then calibrating for one calibrates for all, approximately. For example, sometimes the frequency response (the proportionality constant per frequency) may be known for a new type of device. In such a case, by subtracting the frequency response of one device and adding the frequency response of another, a new device can be calibrated, without ever having tested or used such a device. Such a new device calibration is hereinafter referred to as “a calibration transfer” or “a calibration projection.”

In further embodiments, for example in mobile devices where the headphones jack is also the mic jack, it may be possible to record feedback from the headphones membrane, treating it as a microphone. Specifically, if a user puts a headphone on an ear and the system plays an impulse, the impulse response may be recorded, for example immediately. Similarly, one could sweep through a wide range of frequency (slowly) and get the frequency response. In this way, the characteristics of unfamiliar devices may be acquired.

In accordance with some embodiments, one or more testing protocols may be used as part of the testing hearing.

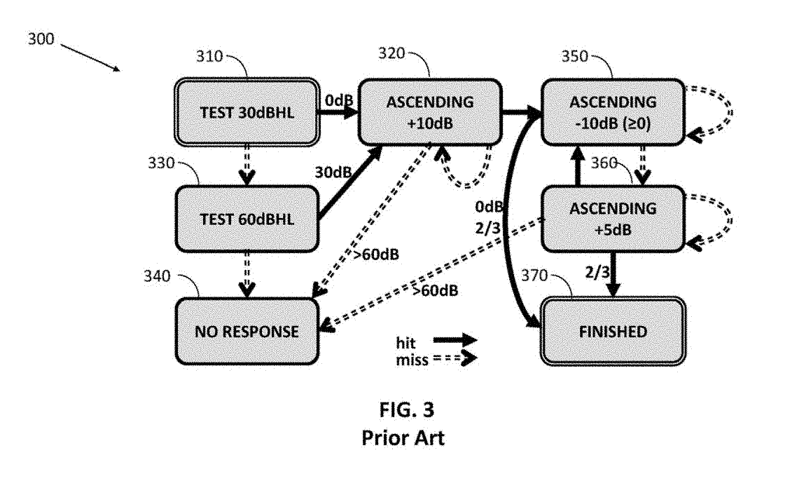

FIG. 3 is a flow diagram showing a prior art example of a testing protocol 300 for a given tone, which may be operated by systems and methods, such as system 100 and/or method 200 in accordance with the prior art. For example the testing protocol 300 may be performed per frequency (per ear) by system 100. In some cases, a calibration process as illustrated in FIG. 2 is performed only once prior to the hearing test. In some instances a calibration process is not performed (e.g. for measuring the calibration transfer) at all. It should be noted that in FIG. 3 all gain levels are in dBHL and gain jumps in dB. First, at step 310 a tone (TEST) at 30 is played. If a hit is identified (a positive indication from the subject), then at step 320 go to ASCENDING (+10) state with gain=0. Otherwise, if no indication received from the subject, at step 330 play a tone (TEST) at 60. If hit, go to step 320 to ASCENDING (+10) state with gain=30. Otherwise, mark “no response” at step 340 and go to next tone. In ASCENDING (+10) state 320, play tone at current gain. If hit, go to DESCENDING state 350 with gain decreased by 10. If miss and gain is less than or equal to 60, go to ASCENDING (+10) 320 state with gain increased by 10. If miss and gain is over 60, mark “no response” 340 and go to next tone. In DESCENDING state 350, play tone at current gain. If hit and gain is positive, go to DESCENDING state with gain decreased by 10. If hit and under 0, go to DESCENDING state with the same gain. If miss go to ASCENDING (+5) state 360 with gain increased by 10. In ASCENDING (+5) state 360 play tone at current gain. If hit, go to DESCENDING state 350 with gain decreased by 10. If miss go to ASCENDING with gain increased by 5. If gain over 60, mark “no response” 340 and go to next tone. At any point keep a table of the hit/miss history per gain level. Upon hit, if two out of latest three tones were marked hit, go to FINISHED state 370 and mark gain.

According to some embodiments, a testing protocol may be based on the Hughson-Westlake (Ascending-Descending/Up-Down) technique, which is a commonly used protocol for testing. Other protocols may also be used. Generally, for each tone, the tone output starts low and is raised (+10 dB jumps) until the patient responds. The tone output is then lowered (−10 dB) until the patient ceases to respond. The tone output is then raised (+5 dB) until the patient responds. Gain level is determined when the level is found at which the patient responds more often than not.

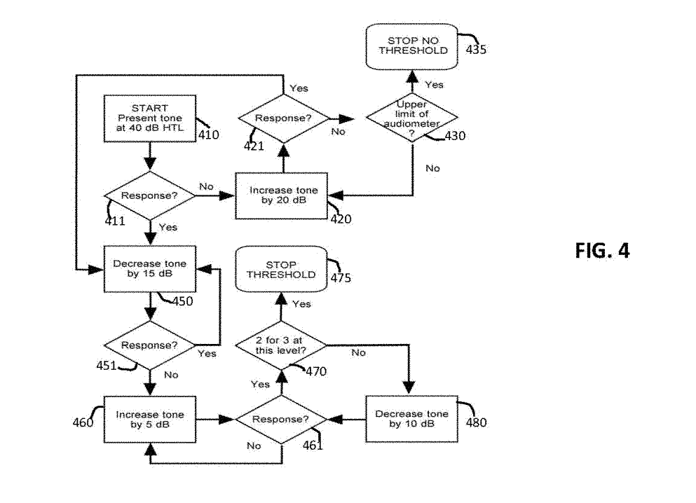

In some embodiments, a test may be designed to be conducted by a state machine, without human judgment being used, for example by a deterministic automaton. As can be seen in FIG. 4, a modified test protocol flow, based on the Hughson-Westlake technique, (known in the art, and adapted from Martia 1983) is shown.

As can be seen with reference to FIG. 4, the test starts with a present tone at 40 db HL (step 410). If there is no response (step 411) the tone is increased by 20 db (step 420). If there is no response (step 421), and this is the upper limit of the audiometer (step 430), then stop the test, there is no threshold to be set (step 435). If there is a response (step 411 or 421), then decrease the tone by 15 db (step 450). Keep decreasing the tone by 15 db until there is no response (step 451), then increase the tone by 5 db 460. Keep increasing the tone by 5 db until there is a response (step 461). If 2 of latest 3 were detected at this level (step 470), stop the test, and establish current gain as threshold (step 475). Otherwise, decrease the tone by 10 dB (step 480), and check response (step 461).

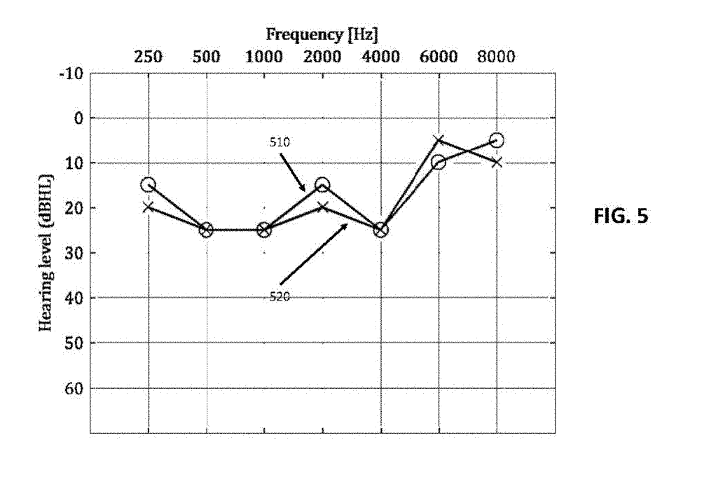

According to some embodiments, an Audiogram Aggregation method is provided, as can be seen with reference to FIG. 5. An audiogram is a conventional description of human hearing. It consists of two charts, or curves, one for each ear, each measured in decibels per frequency, and compared to the hearing of an average young person, which is referred to as 0 dBHL (zero decibel hearing level) in all frequencies. The O's stand for the right ear 510 and X's for the left ear 520. These are standards familiar to all audiologists. The zero level in FIG. 5 is the standard for a young healthy individual as described by ANSI (1996).

The audiogram values indicate how much to amplify at each frequency to get the desired 0 dBHL. Higher values mean greater hearing loss, and more power needed.

An audiometric self-test can be taken many times. By the law of large numbers, it may be assumed that by repeating the test enough times the average will become an increasingly better evaluation of the hearing level (or alternatively, that the error vanishes), therefore precision can be better than that of normal tests taken at very few occasions. This would be true if all samples were taken from the same underlying distribution. However, these assumptions may be challenged by: Human error—which can be attributed to numerous causes; Different environmental noise—depending on location, time of day and specific noise factors (AC, cars, wind, rain, etc.); and fluctuations in hearing level—changes in airflow conduction in the ear, level of awareness, adrenalin, etc.

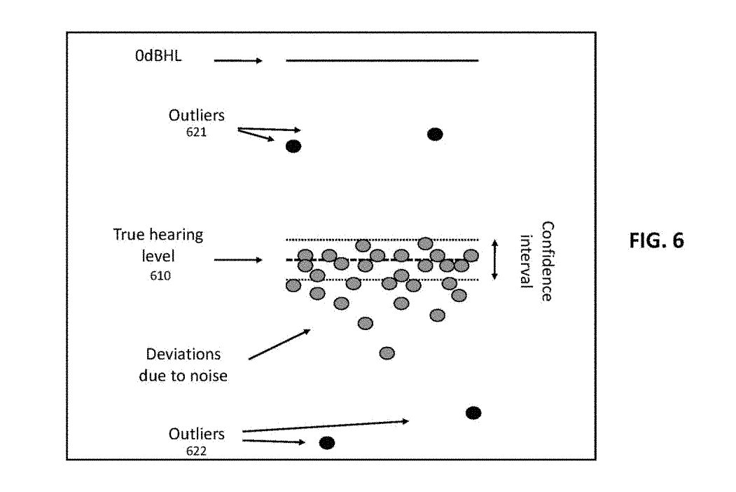

In accordance with some embodiments, as can be seen with reference to FIG. 6, a useful approximation may be generated based on the following guidelines: The user has one true hearing level per frequency, or audiogram A(f) 610; Deviations, AA 620, are a function of the properties of the noise 621, N(t) at the time of recording: X(f)=A(f)+ΔA(f, N(t)); and Deviations that are not a function of the noise (human error, plugged ears, whatever) create outliers 622. Since readings can be as low as the true hearing level, therefore no noise can help us hear better. Therefore, the true or substantially accurate level—the infimum (greatest lower bound) of the measurements, which are not outliers noise, may be evaluated from the readings, and from noise recordings.

According to some embodiments, once a digital audiogram is acquired for a user's device, the user audiogram may be applied for example to a physical layer of a selected device. The selected device may be for example, a smartphone, tablet, computer, television, console, smart speaker etc., comprising one or more processing units configured to integrate the audiograms digital code into the audio output processing, thereby outputting audio in accordance with the user's audiogram.

According to further embodiments, once a digital audiogram is acquired for a user's device, the user audiogram may be applied to a content layer or application layer that is executed by the selected device, for example, YouTube, iTunes, Netflix, audiobooks, radio stations, conferencing software, etc., to enable the application to integrate the audiograms digital code into the audio output, thereby outputting audio in accordance with the user's audiogram.

According to still further embodiments, once a digital audiogram is acquired for a device user, the user audiogram may be applied to a communications layer of communications provider, for example, an ISP, communications provider, infrastructure provider etc., to enable the communications system to integrate filters corresponding to the audiograms into the audio output processing, thereby outputting audio in accordance with the user's audiogram.

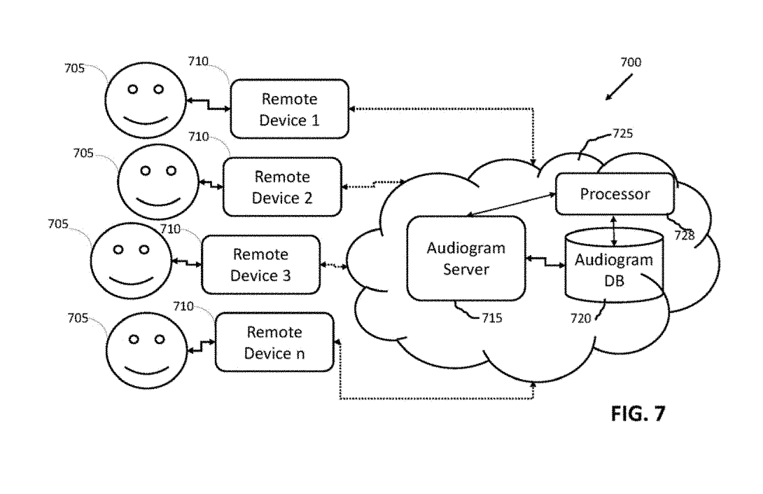

FIG. 7 is a schematic diagram depicting a server-based audiogram analysis engine system 700 for providing an optimized audio for each of a plurality of devices and applications based on an audiogram such as a digital audiogram transmitted via the network to an audiogram server, according to embodiments. In some cases, the system 700 includes multiple users 705, using multiple remote communicating and/or computing devices or systems 710 (such as the device 105 or system 100 of FIG. 1), which may be in communication with an audiogram server 715, communicatively connected to an audiogram database 720 and/or a server processor 728, generally located or connected to a communications cloud 725. In operation, audiogram server 715 runs code, for example executed at processor 728 or at the user's device processor, to enable remote testing of users, optionally using a variety of end user devices. Audiogram server 715 runs further code to analyze user audiogram related data and determine and design the best audio filter for the user. Audiogram server 715 delivers the filter specifications, or embedded code implementing the filter, to multiple applications and/or remote devices 710 that handle digital audio before it is delivered to the user. Audiogram database 720 stores data from multiple users and/or user devices, including user audiogram data, and hardware configurations of user remote devices 710. Further, audiogram database 720 stores data audiogram data from different tests taken at different times and locations, for the purpose of precise evaluation of hearing, and specifically under the influence of different noise profiles. Further, audiogram database 720 can provide the service of a surveillance table, indicating deterioration in hearing before it has manifested to the extent that it is perceived by each user.

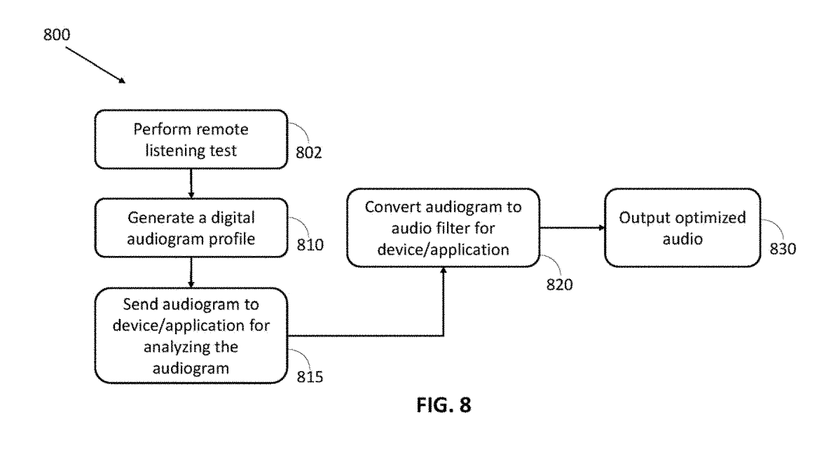

FIG. 8 is a flow diagram 800 of a method for executing of a digital audiogram in an audio playback system, constructed according to embodiments. At step 802, a user performs a remote listening test, to generate at step 810 one or more audiograms such as cloud-based audiograms. In some cases, the remote test may be performed according to method 200 as illustrated in FIG. 2. At step 815 the acquired audiograms are sent to a device and/or application and/or cloud-based audiogram database, for example via a network server for analyzing the audiograms, and/or validate them, and/or update a user hearing or audiogram profile, and/or generate alerts if necessary regarding the user's hearing condition. A user hearing or audiogram profile may include a user's hearing condition and may be stored for example at an audiogram DB such as audiogram DB 720 or at the user's device database. At step 820 the analyzed audiograms are converted into an audio filter specification. In particular, this filter can be a linear infinite/finite impulse response (IIR/FIR) filter specified by a set of coefficients (see for example Szopos et al. 2012). In some cases, other types of filters, linear and non-linear can be used for this purpose. The filter is configured to filter and/or adjust the audio output of the user's selected device audio player for generating an optimized audio output according to the user's audiograms. At step 830 the audio filter calculated in step 820 is applied to audio output from the selected device(s) or application(s).

In some embodiments, additional filters can be applied to the audio output as well, which are not specific to the user, for example, noise reduction for enhanced speech understanding by for example a 300-4000 Hz bandpass filter, or dynamic-range power maximization (Arfin et al. 2009).

In some embodiments, the user's audiogram is integrated into a user audio profile or signature. The user's profile may also include some of the following data: the time and place where tests were conducted, their results, and characteristics of the environmental noise that was present when taken; the time, place, duration, application, and hardware with which a filter was applied; personal information such as age, sex, occupation, and place of residence; and pertinent medical information such as other hearing tests conducted by the user, and information regarding the user's hearing aids.

In some embodiments, the user's audiogram is generated in an audiogram digital format that may be executed by multiple vendors in external devices, programs, applications, etc.

In some embodiments, the user's audiogram may include personal preferences, for example, listening preferences for different types of audio etc.

According to certain embodiments, a user audio output device can be used to optimize phone calls and other mobile audio output to a user, from a phone device, audio device, earphones etc. For example, if a user A using a device in accordance with embodiments is carrying out a conversation with another user B, and even if user B is not using a supported device capable of audio filtering, in accordance with embodiments the audio output from user A's device is optimized for user B's hearing, according to user B's audiogram, as recorded in the Audiogram Database 720.

According to certain embodiments, a user audio output device can share audiometric data with other devices (TV, PC, car stereo, etc.) if they support a standard interface for audio equalization. In some cases, the audiometric data may include the user's audiogram, a filter specification, or preferences related to the user's hearing.

According to some embodiments, the user's audiogram may be integrated into a processor of noise cancellation earphones, thereby enabling earphones to be used as audio enhancing devices and/or as hearing aid devices. For example, audio sounds received may be filtered and optionally re-generated with filtered audio signals, amplified signals etc., to enable transmitting of personalized audio output.

Further, since the user's audiogram is cloud based, it may be integrated into multiple connected devices and systems, to allow seamless application of personalized audio across different devices, anywhere.

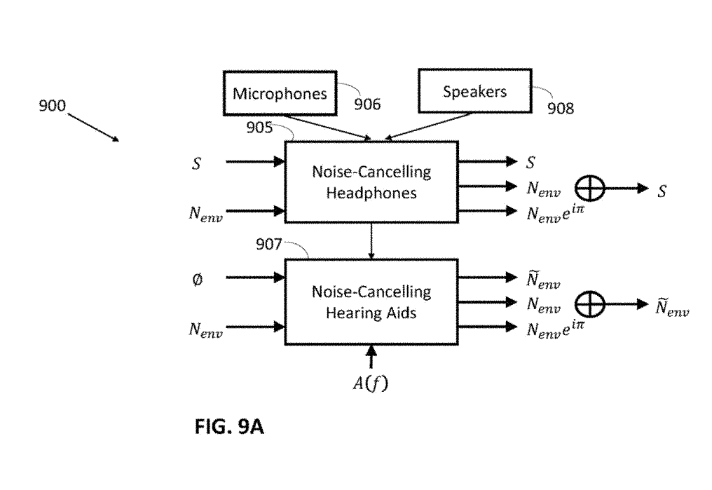

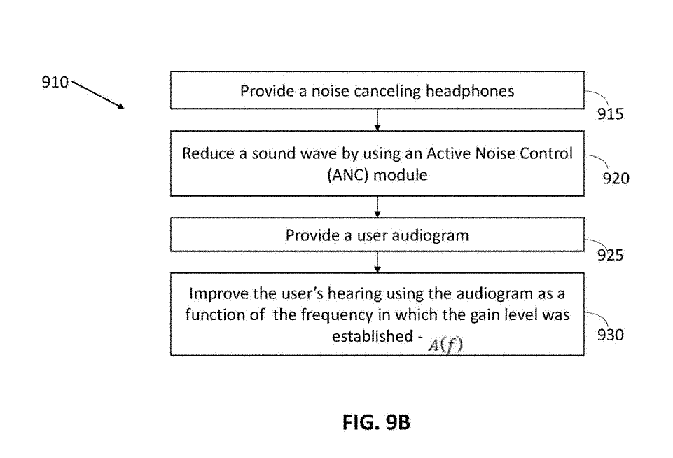

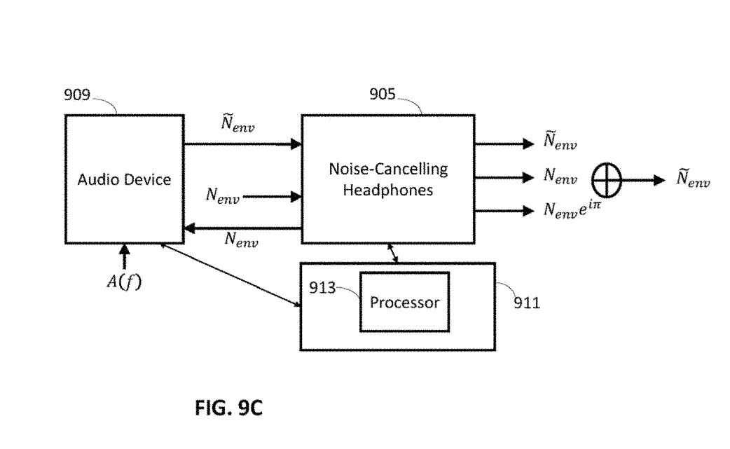

In accordance with some embodiments, there are provided methods and systems for transforming Noise-Cancelling Headphones to Noise-Cancelling Hearing Aids, as described with reference to FIGS. 9A-9C.

Those of skill in the art will also recognize that suitable noise canceling headphone 905 may be, by way of non-limiting examples a SONY® WH-1000XM2, or Philips® Fidelio NC1, Bose® QuietComfort 35, or the like.

According to some embodiments, the noise-cancelling headphone may be the noise-cancelling headphone described in U.S. Pat. No. 8,045,726, incorporated herein by reference. For example the noise-cancelling headphone may include a cancel signal generator that receives ambient noise via an electro-acoustic transducer and generates and outputs a cancel signal eliminating the noise, and a speaker unit that outputs an audio signal and a cancel signal, and connects the cancel signal generator to a first terminal of two input terminals of the speaker unit and connects a sound source of an audio signal to a second terminal thereof, whereby obtaining the noise-cancelling headphone with which one can enjoy music with high quality without the change in the sound quality and volume between when a noise-cancelling function is activated and when deactivated.

In some cases, the noise-cancelling headphones 905 is connectable to a portable computing platform having a processor and one or more microphones 906 and/or speakers 908.

A noise canceling hearing aid may be defined, in some embodiments as a device configured to adjust, rather than amplifies, environmental sound to the user. It is different from a conventional hearing aid because it attenuates (or cancels) the original environmental audio actively, rather than passively blocking it (as a hearing aid typically does), and additionally emits the same audio, with necessary adjustments specific to the user. Equivalently, it can be stated that, due to the well-known superposition principle, a noise canceling hearing aid emits the difference between the original ambient signal and the adjusted signal, added in-phase to the original ambient signal.

FIG. 9A illustrates a noise canceling headphones 905 transformed to a noise canceling hearing aid 907, according a transformation method 910 illustrated in FIG. 9B.

At step 915 the noise canceling headphones 905 is provided. At step 920, an Active Noise Control (ANC) module is used for reducing a sound wave, for example by superimposing an additional source with equal amplitude and inverted phase at all times. For example, the Noise-cancelling headphones 905 may employ the ANC method to reduce a sound originating from the environment and perceived by the user wearing them. The method includes for example, superimposing a desired signal S on a signal emitted by the speakers. The desired effect is to replace the environmental noise Nₑₙᵥ with a signal S chosen by the user, e.g. music from an audio player. To achieve this, in some embodiments, the noise-cancelling headphones 905 may be connected to one or more microphones 906 and speakers 908 for example at each ear, and real-time processing, necessary for ANC, is used to relay the sound picked-up by each microphone and mix it (in antiphase) with the signal.

At step 925 a user audiogram, such as a digital audiogram is provided to the noise cancelling headphones, for example as illustrated in FIG. 8. At step 930, the user's audiogram is used with the noise-cancelling headphones 905 to optimize the user's hearing by applying the gain levels recorded in the user's audiogram for each frequency, in the following manner: the audiogram, which functions as a characterization of a person's threshold hearing gain level in units of dBHL, is marked A(f), where f is the frequency in which the gain level was established. Further, a signal \tilde{S} is denoted as the signal S filtered by a filter applying a gain at each frequency equivalent to A(f), for example using an finite impulse response (FIR) filter. Additionally, by dropping the input signal S, and replacing the output with the filtered environmental noise, the method effectively replaces sound input from the environment with appropriately filtered sound.

In some embodiments, as illustrated in FIG. 9C, the above described process may be applied to multiple hardware devices or systems, without requiring any change to the hardware configuration of these devices or systems. For example, the noise cancelling headphones may be connectable to an audio device 909 and to a portable computing platform 911, such as a mobile phone having a processor 913. In such cases, an appropriate software driver, executed for example by the processor 913 may include instructions for providing a signal input S, to enable the device 909 to acquire the noise detected by the headphones microphones Nₑₙᵥ, The device can then filter this signal and deliver Ñₑₙᵥ as the input signal to the headphones. The result in some cases is equivalent to the proposed noise-cancelling hearing aid system, but no additional hardware is required.

According to some embodiments, a user audiogram can be applied to a vehicle audio output device or system, to provide enhanced audio output for a driver or user of the vehicle or transporter.

According to some embodiments, a user audiogram can be applied to call center or other typically noisy environment, to optimize audio output to users in the noisy environment.

According to some embodiments, a user audiogram can be applied to mobile communication devices, such as phones, smart phones, tablets, wearable devices etc., to enhance phone call and other audio output quality.

According to some embodiments, a user audiogram can be applied to mobile communication devices, such as phones, smart phones, tablets, wearable devices etc., to enhance music or other audio output quality.

According to some embodiments, a user audiogram can be applied to multiple music or other audio related applications running on a computer or communications devices, such as music playing programs, audiobook players, Podcasts players, games etc.

According to some embodiments, a user audiogram can be applied to televisions, computer screens, consoles, and other entertainment systems or devices, optionally applying audio optimization to directional speakers.

According to some embodiments, a user audiogram can be applied to computers, PCs, mobile devices, televisions and other screening devices running audio-based content programs or applications, to enable optimized audio output for content applications such as YouTube, Netflix, cable TV, Spotify, Apple music, online radio stations, games etc.

According to some embodiments, a user audiogram can be applied to a cloud-based Conference Call program, to optimize output to conference call users.

According to some embodiments, a user audiogram can be applied to a smart home or other smart environments that integrate audio output. In some examples, a user audiogram can be applied to digital assistants such as Alexa, Seri, and other personal robots, guides, assistants, to optimize communication and/o content output for user(s).

According to some embodiments, a user audiogram can be applied to broadcast radio, typically on the radio player hardware.

According to some embodiments, a user audiogram can be applied to an audio output channel, device, application etc., to provide audio output adapted to a user's accent and/or dialect.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

The present disclosure provides computer control systems that are programmed to implement methods of the disclosure. FIG. 10 shows a computer system 1001 suitable for incorporation with the methods and apparatus in accordance with some embodiments of the present disclosure. The computer system 1001 can process various aspects of information of the present disclosure, such as, for example, questions and answers, responses, statistical analyses. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., a parent). Examples of remote computer systems and mobile communication devices include personal computers (e.g., portable PC), slate or tablet PC's (e.g., Apple® iPad, Samsung® Galaxy Tab), telephones, Smart phones (e.g., Apple® iPhone, Android-enabled device, Blackberry®), personal digital assistants, wearable medical devices (e.g., Fitbits), or medical device monitors (e.g., seizure monitors). The user can access the computer system 1001 with the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, questions and answers, analysis results, recommendations. Examples of UI's include, without limitation, a graphical user interface (GUI) and web-based user interface.

Methods and systems of the present disclosure can be implemented by way of one or more algorithms and with instructions provided with one or more processors as disclosed herein. An algorithm can be implemented by way of software upon execution by the central processing unit 1005. The algorithm can be, for example, random forest, graphical models, support vector machine or other.

Although the above steps show a method of a system in accordance with an example, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

Each of the examples as described herein can be combined with one or more other examples. Further, one or more components of one or more examples can be combined with other examples.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the method and apparatus of the present disclosure provided herein without departing from the spirit and scope of the invention as described herein.

While preferred embodiments of the present disclosure have been shown and described herein, it will be obvious to those skilled in the art that such embodiments are provided by way of example only. Numerous variations, changes, and substitutions will be apparent to those skilled in the art without departing from the scope of the present disclosure. It should be understood that various alternatives to the embodiments of the present disclosure described herein may be employed without departing from the scope of the present invention. Therefore, the scope of the present invention shall be defined solely by the scope of the appended claims and the equivalents thereof.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

We claim:

1. A system for providing optimized audio output, the system comprising:
a device connectable to one or more earphones, the device comprising:
a computing platform having a processor;
an audio unit configured to generate one or more output signals of arbitrary amplitude to the one or more earphones;
at least one microphone configured to record the power level of said outputs signals and calculate a proportionality constant for each frequency of said output signals; and wherein the processor is further configured to:
analyze the proportionality constant for each frequency of one or more feedback signals from said one or more earphones to yield calibration data;
adjust the amplitude or frequency based at least on the calibration data to calibrate the device;
generate one or more audiograms resulted by conducting a hearing test using the calibrated device;
adjust said device power level according to said received one or more audiograms.

2. The system of claim 1 wherein said processer is configured to calculate a fractional amplitude coefficient for each of said feedback signals for providing said calibration data.

3. The system of claim 1 wherein said hearing test is executed by said processor according to a state deterministic automaton.

4. The system of claim 1 wherein said hearing test is based on the Hughson-Westlake technique.

5. The system of claim 1 wherein said audiograms are applied to one or more selected remote devices having a processor, said processor is configured to adjust said selected device audio power based on said audiograms.

6. The system of claim 1 wherein said device is a mobile communication device comprising wireless communication circuitry to communicate with a remote server, and wherein the processor comprising instructions to transmit the audiogram to the remote server.

7. The system of claim 6 wherein in response to said instructions the audiograms are further integrated in said remote server database for adjusting an audio output control of one or more contents or application in said remote server database in accordance with the integrated audiogram.

8. The system of claim 7 wherein said audiograms are applied to a communication layer of communications provider of said remote server for adjusting an audio output control of said communication layer of communications provider in said remote server database in accordance with the integrated audiogram.

9. The system of claim 8 wherein said content or application is selected from the group consisting of: YouTube, iTunes, Netflix, audiobooks, radio stations, conferencing software.

10. The system of claim 1 wherein said one or more earphones are selected from a group consisting of: noise cancellation earphones, wireless earphones, wired earphones.

11. The system of claim 1 wherein said at least one microphone is a sound level meter (SLM).

12. The system of claim 1 wherein said audiograms are generated in a digital format.

13. The system of claim 1 wherein said audiograms comprise personal preferences of said user.

14. The system of claim 1 wherein said audiograms are shared with other devices or applications.

15. The system of claim 14 wherein said other devices or applications are selected from the group consisting of: computers, PCs, mobile devices, televisions YouTube, Netflix, cable TV, Spotify, Apple music, online radio stations, games.

16. The system of claim 1 wherein said audiograms are shared with other devices via a network server.

17. The system of claim 1 wherein said audiograms are applied to a cloud-based Conference Call program, to optimize output to conference call users.

18. The system of claim 1 wherein said audiograms are applied to digital assistant devices.

19. The system of claim 18 wherein said digital assistant devices are one or more of Alexa, Seri, personal robots, guides, assistants.

20. The system of claim 1 wherein said audiograms are applied to broadcast radio.

21. The system of claim 1 wherein said audiograms are integrated into a processor of noise cancelation earphones for converting said noise cancelation earphones to audio enhancing devices or a hearing aid devices, and wherein said conversion comprises filtering audio signals received at said noise cancelation earphones and amplifying audio signals yield a personalized audio output by said noise cancelation earphones.

22. A method for providing optimized audio output using a device comprising an audio unit and a processor, and wherein the device is further connectable to an earphone, the method comprising:
performing a hearing test to a user using said device, the hearing test comprising generating signals at selected frequencies and hearing levels and recording the user feedback;
generating a digital audiogram profile based on said hearing test;
transmitting said digital audiogram to said device or to a remote server;
converting the digital audiogram to an audio filter;
adjusting said device audio output to yield an optimized audio output.

23. The method of claim 22 wherein converting the digital audiogram to an audio filter comprises shaping input signal so as to be amplified by a magnitude equivalent to the audiogram's gain level for each tested frequency.

24. The method of claim 23 wherein the frequency response derived from said digital audiogram is 50% the gain levels.

25. The method of claim 22 wherein adjusting said device audio output is configured by applying said audio filter, or other filters which may be beneficial for the user, such as noise reduction, or band-pass filtering.

26. A server-based audiogram analysis engine system, the system comprising:
a remote server, said remote server is in communication with a database and a remote processor;
a plurality of remote devices connectable respectively to a plurality of earphones, wherein each of said plurality of remote devices comprises:
a computing platform having a processor;
an audio unit configured to generate respectively one or more output signals of arbitrary amplitude to said plurality of headphones;
wireless communication circuitry to communicate with said remote server;
and wherein each of said remote devices is configured to:
perform a hearing test to one or more users, the hearing test comprises:
generating one or more output signals of arbitrary amplitude to yield an audiogram profile respectively for each of said users;
transmit said audiogram profile to said remote server, wherein said remote server comprises instructions to:
analyze said audiogram profiles;
convert said audiogram profiles to yield a personalized audio filter for each of said plurality of remote devices.

27. The server-based audiogram analysis engine system of claim 26 wherein said analysis comprises generating one or more alerts.
